(19) United States
(12) Patent Application Publication
(10) Pub. No.: US 2013/0197410 A1
(43) Pub. Date: Aug. 1, 2013

(54) SELF-ACTIVATED POSTURAL COMPLIANCE LIFT-ASSISTANCE DEVICE

(71) Applicants: Sean Petterson, Mount Sinai, NY (US); Justin Lamont Hillery, Rochester, NY (US)

(72) Inventors: Sean Petterson, Mount Sinai, NY (US); Justin Lamont Hillery, Rochester, NY (US)

(73) Assignee: STRONG ARM INC., Rochester, NY (US)

(21) Appl. No.: 13/878,914

(22) PCT Filed: Mar. 30, 2012
(86) PCT No.: PCT/US12/31440

§ 371 Date: Apr. 11, 2013

Related U.S. Application Data
(60) Provisional application No. 61/516,277, filed on Apr. 1, 2011.
(60) Provisional application No. 61/595,187, filed on Feb. 6, 2012.

(51) Int. Cl. A61F 5/02

(52) U.S. Cl. CPC A61F 5/02
USPC 602/19

(57) ABSTRACT

The present invention preferably relates to a self-activated postural compliance lift-assistance device that puts the wearer in an increasingly supported lifting posture, thereby providing a lift-assistance device that conforms with best ergonomic practices for lifting.

### CROSS-REFERENCE TO RELATED APPLICATIONS

This application claims the benefit of PCT filing Serial No. PCT/US2012/031440, filed Mar. 30, 2012, U.S. provisional filing Ser. No. 61/516,277, filed Apr. 1, 2011, and U.S. provisional filing Ser. No. 61/595,187, filed Feb. 6, 2012. The contents of these related applications are herein incorporated in their entireties by reference.

### FIELD OF THE INVENTION

The present invention preferably relates to a self-activated postural compliance lift-assistance device that puts the wearer in an increasingly supported lifting posture, thereby providing a lift-assistance device that conforms with best ergonomic practices for lifting.

### BACKGROUND OF THE INVENTION

According to the U.S. Occupational Health and Safety (“OSHA”) technical manual, “back disorders can develop gradually as a result of microtrauma brought about by repetitive activity over time or can be the product of a single traumatic event . . . acute back injuries can be the immediate result of improper lifting techniques and/or lifting loads that are too heavy for the back to support.” See OSHA technical manual, Section VII, Chapter I, “Back Disorders and Injuries,” available at osha.gov/dts/osta/otm/otm_vii/otm_vii_{—}1.html#app_vii:1_{—}2 (“OSHA Manual”). As the OSHA Manual then goes on to note, “although back injuries account for no work-related deaths, they . . . are one of the leading causes of disability for people in their working years and afflict over 600,000 employees each year with a cost of about $50 billion annually in 1991 according to NIOSH . . . [and] the frequency and economic impact of back injuries and disorders on the work force are expected to increase over the next several decades as the average age of the work force increases and medical costs go up.”

Given the enormous health and economic consequences of lifting-related back injuries, there have been a large number of devices developed that purport to be useful for better lifting safety. See, e.g., the numerous examples of such devices within U.S. Classification Class/Subclass 602/19. However, in 1994 a “Back Belt Working Group” of the National Institute of Occupational Health and Safety (“NIOSH”) reviewed commercially available lifting belts and concluded that such “back belts do not mitigate hazards to workers posed by repeated lifting, pushing, pulling, twisting, or bending” and that, in light of “insufficient data indicating that typical industrial back belts significantly reduce the biomechanical loading of the trunk during manual lifting,” this working group concluded that 1) back belts are not recommended for preventing injuries; and, 2) back belts are not personal protective equipment (“PPE”). See NIOSH publication 94-122, available at cdc.gov/niosh/docs/94-122/ (“NIOSH1994”). See also NIOSH's 1996 summary of these results, NIOSH publication 94-127, October, 1996, available at cdc.gov/niosh/docs/94-127/ (“NIOSH 1996”).

In light of the above health and economic consequences of lifting-related back injuries and the lack of suitable devices for preventing such injuries, there is a great need for the development of better lift-assistance devices.

### SUMMARY OF THE INVENTION

The present invention relates to a self-activated postural compliance lift-assistance device that puts the wearer in an increasingly supported lifting posture, thereby providing a lift-assistance device that conforms with best ergonomic practices for lifting.

In embodiment 1, the present invention is directed to a lift-assistance device comprising: a load transfer means (“LTM”), for transferring the load weighting from the lifting point over the shoulders and down to the lower torso; a postural compliance means (“PCM”), for passively/actively enforcing the appropriate back posture; and, a coupling means (“CM”), for coupling increased loading on the load-transfer means into increasing engagement of the postural compliance means.

In embodiment 2, the present invention is directed to the lift-assistance device of embodiment 1, where the appropriate back posture for each engagement level of the postural compliance means is one that promotes maintenance of the natural curve of the back at that engagement level of the postural compliance means.

In embodiment 3, the present invention is directed to the lift-assistance device of embodiment 1, where the appropriate back posture for each engagement level of the postural compliance means is one that reduces peak lumbar flexion at that engagement level of the postural compliance means.

In embodiment 4, the present invention is directed to the lift-assistance device of embodiment 3, where the reduction in peak lumbar flexion at a particular weight lifted is at least as shown in FIG. 15 for that weight.

In embodiment 5, the present invention is directed to the lift-assistance device of embodiment 1, where the appropriate back posture for each engagement level of the postural compliance means is one that promotes a measurable reduction in wearer injuries.

In embodiment 6, the present invention is directed to the lift-assistance device of embodiment 5, where the measurable reduction in wearer injuries is a measurable reduction in wearer back injuries.

In embodiment 7, the present invention is directed to the lift-assistance device of embodiment 1, further comprising a lift coupling means (“LCM”) for each arm, where each LCM transfers at least part of the weight of the load to be lifted to the LTM for that arm.

In embodiment 8, the present invention is directed to the lift-assistance device of embodiment 7, where the LCM is selected from the group consisting of gloves, hooks, grippers and gripping strips such as Velcro®.

In embodiment 9, the present invention is directed to the lift-assistance device of embodiment 1, where the CM is one or more of the CM exemplified in FIGS. 7-9.

In embodiment 10, the present invention is directed to the lift-assistance device of embodiment 1, where increasing engagement of the postural compliance means is linearly related to the weight supported by the LTM.

In embodiment 11, the present invention is directed to the lift-assistance device of embodiment 1, where increasing engagement of the postural compliance means is non-linearly related to the weight supported by the LTM.

In embodiment 12, the present invention is directed to the lift-assistance device of embodiment 11, where increasing engagement of the postural compliance means is a bi-state engagement from disengaged (state 1) to fully engaged (state 2).

In embodiment 13, the present invention is directed to the lift-assistance device of embodiment 1, where the device additionally includes one or more sensors for assaying one or more of the loads being lifted, loading at one or more points on the user's body or one or more indicators of strain on the user's body from lifting.

In embodiment 14, the present invention is directed to the lift-assistance device of embodiment 13, where the one or more sensors includes one or more unsafe-weight sensors.

In embodiment 15, the present invention is directed to a method for reducing lifting-related injuries comprising lifting while wearing a lift-assistance device comprising: a load transfer means (“LTM”), for transferring the load weighting from the lifting point over the shoulders and down to the lower torso; a postural compliance means (“PCM”), for passively/actively enforcing the appropriate back posture; and, a coupling means (“CM”), for coupling increased loading on the load-transfer means into increasing engagement of the postural compliance means.

In embodiment 16, the present invention is directed to the lift-assistance device of embodiment 15, where the appropriate back posture for each engagement level of the postural compliance means is one that reduces peak lumbar flexion at that engagement level of the postural compliance means.

In embodiment 17, the present invention is directed to the lift-assistance device of embodiment 16, where the reduction in peak lumbar flexion at a particular weight lifted is at least as shown in FIG. 15 for that weight.

In embodiment 18, the present invention is directed to the lift-assistance device of embodiment 15, where the appropriate back posture for each engagement level of the postural compliance means is one that promotes a measurable reduction in wearer injuries.

In embodiment 19, the present invention is directed to the lift-assistance device of embodiment 15, further comprising a lift coupling means (“LCM”) for each arm, where each LCM transfers at least part of the weight of the load to be lifted to the LTM for that arm.

In embodiment 20, the present invention is directed to the lift-assistance device of embodiment 19, where the LCM is selected from the group consisting of gloves, hooks, grippers and gripping strips such as Velcro®.

In embodiment 21, the present invention is directed to the lift-assistance device of embodiment 15, where the CM is one or more of the CM exemplified in FIGS. 7-9.

### BRIEF DESCRIPTION OF THE DRAWINGS

The drawings provided in the present invention are provided solely to better illustrate particular embodiments of the present invention, and specifically do not provide an exhaustive or limiting set of embodiments of the present invention.

FIG. 1 provides a schematic example of non-ergonomic lifting, i.e., lifting by keeping the legs straight/locked and bending at the waist with a hunched back.

FIG. 2 provides a schematic example of ergonomic lifting, which involves keeping the weight as close to the body as possible, keeping the torso relatively erect to preserve the natural curvature of the spine, and using the leg muscles to do the lifting, e.g., by going from a squat to a standing position.

FIG. 3 provides a schematic example of a simple device intended to put the user in an appropriate lifting posture. As long as the wearer keeps his/her back relatively erect, increasing loading on the two straps will pull the user further upright, that is, into the appropriate conformation for lifting.

FIG. 4 shows that a device as simple as that shown in FIG. 3 will not function appropriately because the user will naturally tend to hunch over, thereby worsening the wearer's posture and putting even greater loading on his/her spine

FIG. 5 provides a schematic example of one non-limiting embodiment of the present invention directed to a device that has at least the additional functionality of either preventing hunching over or encouraging erect posture, or a combination of the two. The view in this figure is of the back of the torso.

FIG. 6 provides a more generalized schematic example of an embodiment of the invention in which the load-transfer means LTM (e.g., straps S1 and S2) transfers the load from the lifting point over the shoulders and down to the waist belt W, where the weight is then transferred via coupling means C to the postural compliance means PCM, which upon increased loading increasingly engages to ensure the appropriate lifting posture of a non-loaded curve of the spine and prevents/enforces non-hunching.

FIG. 7 provides a schematic of a different embodiment of the invention with the LTM, PCM and C means described above; in this embodiment there is a single coupling means C that rides in a vertical channel in slide SL, where motion of C vertically in the channel of slide SL results in the coupling of increased weight on the load-transfer means LTM to increasingly enforced postural compliance via tightening of the PCM.

FIG. 8 provides a photograph of a prototype lifting vest of the embodiment of the present invention shown schematically in FIG. 7.

FIG. 9 provides an exploded view of the coupling means C in the channeled slider SL of the embodiment shown in FIG. 7.

FIGS. 10-13 show various additional exemplary embodiments of the present invention.

FIG. 14 provides a super-positioning of images obtained at various stages during the lifting process of a user wearing one embodiment of the present invention.

FIG. 15 provides data on lumbar kinematics during weight-lifting without and with an exemplary lift-assistance device of the present invention. These data show that the lift-assistance device has a significant effect on reducing peak lumbar flexion during lifting, with the reduction seen for heavier loads reaching over 50%.

FIGS. 16-17 show various embodiments of the “load coupling means” of the present invention.

FIGS. 18-20 show one embodiment of the “load-activated grip-assisting glove” embodiment contemplated in the present invention.

### DETAILED DESCRIPTION OF THE INVENTION

Note that in the present invention, “a” or “an” are explicitly not limited to the singular form; instead, “a” and “an” are explicitly intended to be synonymous with “at least—but not limited to—one instance of” the term being referenced.

### Appropriate Lifting Posture/Sequence of Lifting Postures

The present invention is based on the recognition that lifting-related injuries can be significantly reduced by: 1) compliance with the appropriate sequence of postures during lifting; and, 2) mechanical distribution of weight across the body as determined by ergonomic studies. In order to implement 1) and 2) above the present invention is particularly directed to an unconventional device for insuring sequenced postural compliance and appropriate weight distribution, while also supplying a third critical factor of 3) a device design that is comfortably donned/removed and worn, in order to prevent user non-compliance, within 4) appropriate manufacturability parameters, e.g., durability and price.

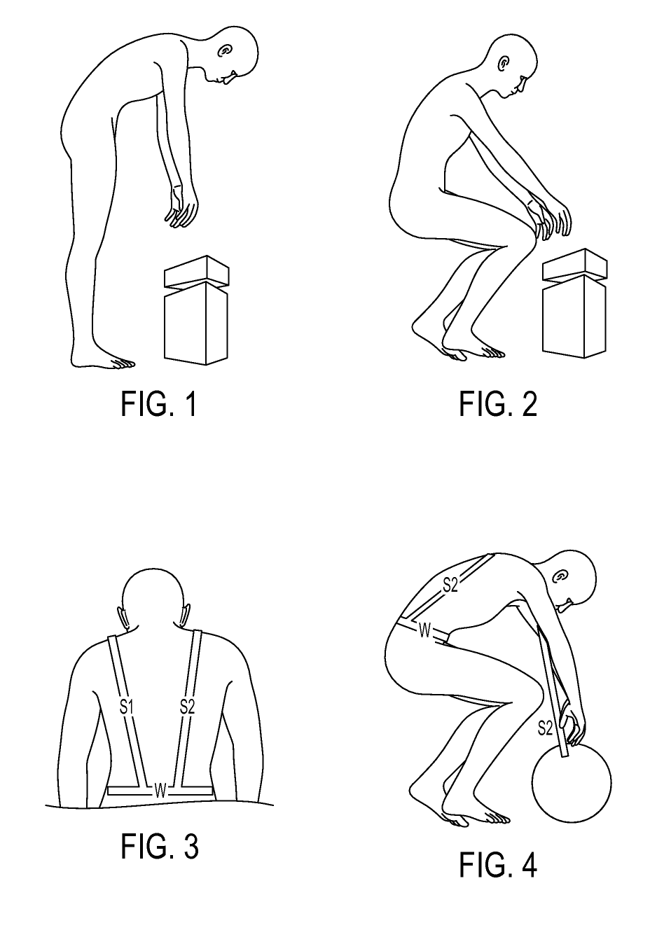

With regard to the first factor, the appropriate sequence of postures during lifting, a large number of ergonomic studies have established a standard sequence of postures for lifting. The Mayo Clinic, for example, lists a lifting sequence consisting of 6 steps: 1) start in a safe position; 2) maintain the natural curve in your lower back; 3) use your legs; 4) squatting instead of kneeling; 5) let your legs do the work; and, 6) avoid twisting. See mayoclinic.com/health/back-pain/LB00004_D. This sequence of steps captures the two basic principles of a) not lifting at the waist, and instead b) lifting with the back relatively erect, using the legs. Thus as shown in FIG. 1, lifting by keeping the legs straight/locked and bending at the waist with a hunched back is non-ergonomic lifting, since lifting in this posture forces the spine to support both the weight of the upper body and the weight of the load being lifted and, worse, the distance of the load out from the center of the body in this posture enormously increases the strain acting on the spine, e.g., into the thousands of foot-pounds of torque. See, e.g., “Biomechanics of Safe Lifting, available at ergo.human.cornell.edu/DEA3250notes/lifting.html. See also, e.g., “Applications Manual for the Revised NIOSH Lifting Equation,” 1994, NIOSH publication PB94-176930.

Instead, as shown in FIG. 2, ergonomic lifting involves keeping the weight as close to the body as possible, keeping the torso relatively erect to preserve the natural curvature of the spine, and using the leg muscles to do the lifting, e.g., by going from a squat to a standing position. In this regard, it is worthwhile nothing that, according to NIOSH1996, “[i]t would appear that abdominal belts help restrict the range of motion during side to side bending and twisting. However, they do not have the same effect when the worker bends forward, as in many industrial lifting situations.” Thus it would appear that current support belts generally have little if any effect on ensuring this correct lifting posture and, as a result, a worker wearing a conventional lifting belt is unlikely to adopt this posture—keeping the weight close to the body by minimizing bending from the waist (thereby keeping the torso upright and lowering the stresses on the spine)—or the coupled requirement for proper lifting of using the legs to lift, i.e., by going from a squat to an erect position during lifting.

### The Lift-Assistance Device of the Present Invention

In this regard, in one aspect the present invention is directed to ensuring that a wearer appropriately lifts loads 1) with the back in a series of positions that—as the loading increases—becomes increasingly constrained to be erect (i.e., a “self-activated” device), thereby ensuring that the user's spine experiences minimized loading during lifting and particularly when lifting the full load. Referring to FIG. 3, a simple mechanical device for achieving this purpose might consist of two straps (S1 and S2) attached at a waist belt (W) and going over one or more—and preferably both—of the two shoulders down to the weight being lifted in front, with the distal (far) ends of these straps ending either at the hands or in hooks or other grips that directly contact the weight being lifted. As long as the wearer keeps his/her back relatively erect, increasing loading on the two straps will pull the user further upright, that is, into the appropriate conformation for lifting.

In reality, a device as simple as that shown in FIG. 3 will not function appropriately because the user will naturally tend to hunch over, thereby worsening the wearer's posture and putting even greater loading on his/her spine, a situation that is shown in FIG. 4. Note that the weight in this figure is shown as being a sphere; also, S2 is shown as extending down directly to the weight, although this is only one embodiment of the present invention. S1 is not shown in this figure, but will have a form similar to that of S2.

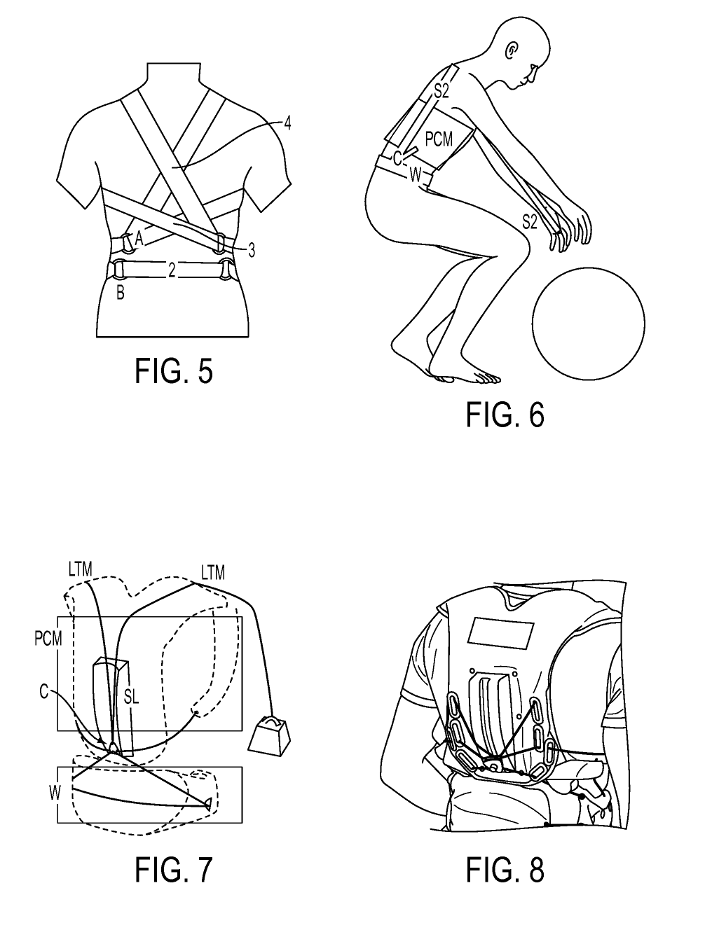

In order to prevent the situation depicted in, e.g., FIG. 4, in one embodiment the present invention is directed to a device that has at least the additional functionality of either preventing hunching over or encouraging erect posture, or a combination of the two. FIG. 5 provides one embodiment of such a device. Specifically, FIG. 5 shows two shoulder straps 4 (for clarity, only the shoulder strap crossing the left shoulder is labeled in this schematic, but the designation refers to both straps) that in this embodiment criss-cross the shoulders and descend across the back to D-ring “coupling means” (A; although only the D-ring on the left side of the wearer's body is labeled in this figure, the designation also refers to the corresponding D-ring on the right side) or other (non-D-ring) forms of coupling means that allow the shoulder straps 4 to descend down to or near to the waist (in the embodiment of FIG. 4 the D-ring coupling means A are fixed in position directly above the hip/waist belt 2; in general the invention contemplates one or more attachment points on the torso, preferably the lower torso, and still more preferably in the region of the waist) and that, under loading, allow the continuation of these shoulder straps 3 to slide through these D-ring coupling means A. Since these continuation straps 3 continue on around the body where they are fixed (these ends are not shown in the figure), loading on straps 4 results in tensioning of straps 3 through sliding of the straps through the coupling means A, with the tensioning of straps 3 compressing the torso so as to support/alter the wearer's position to a more erect posture, thereby ensuring postural compliance that prevents the situation shown in FIG. 4.

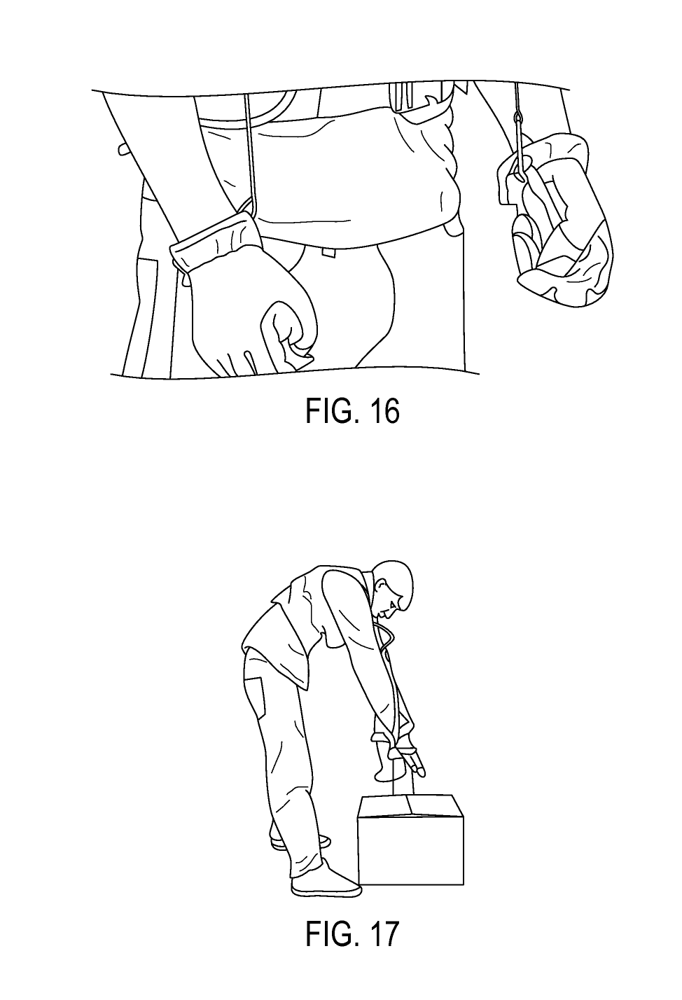

FIG. 6 shows a more generalized schematic representation of this embodiment of the present invention, where the straps S1 and S2 (only S2 is shown; S1 is the mirror image of S2, in that it attaches in the region of the right hand and crosses the left shoulder in this criss-crossed S1/S2 embodiment) ascend from an attached positioning at the “lifting point” in the region of the lower forearm/hands (e.g., by “lift coupling means” such as gloves, lifting hooks, wrist-straps, etc., as described in more detail below and in, e.g., FIGS. 16-17), over the shoulders (in either crossed or uncrossed conformations) and down across the back to the waist belt W, where the straps are connected via coupling means C (here on each side of the body) to the postural compliance means PCM of the apparatus. In this representation, the coupling means C for each strap S1 and S2 could be the D-ring coupling means of FIG. 5, although other coupling means are contemplated (see below). The postural compliance means PCM could be, e.g., the straps 3 of FIG. 5 that compress the torso upon loading of the straps, although this is only one of the embodiments contemplated for the postural compliance means PCM.

Thus in the embodiment depicted in FIG. 6, there are three critical sections to the embodiment: 1) the load-transfer means LTM, e.g., straps S1 and S2 (although the term “LTM” encompasses fewer or more straps, non-strap means such as ropes or strings, etc.), which transfers the load weighting from the lifting point (hands, wrists, forearms, etc.) over the shoulders and down to the lower torso, typically the waist belt W (again, the invention most generally contemplates one or more attachment points on the torso, preferably the lower torso, and still more preferably in the waist region); 2) the postural compliance means PCM, which upon increased loading increasingly engages to ensure the appropriate lifting posture of a non-loaded curve of the spine and prevents/enforces non-hunching (i.e., prevents the inappropriate back position of FIG. 4); and, 3) coupling means C (multiple coupling means are shown in this figure, but other numbers of such coupling means are contemplated, as in, e.g., FIG. 7), which allows increased loading on the LTM such as S1/S2 to be translated into increasing engagement of the PCM and, therefore, increasing postural compliance.

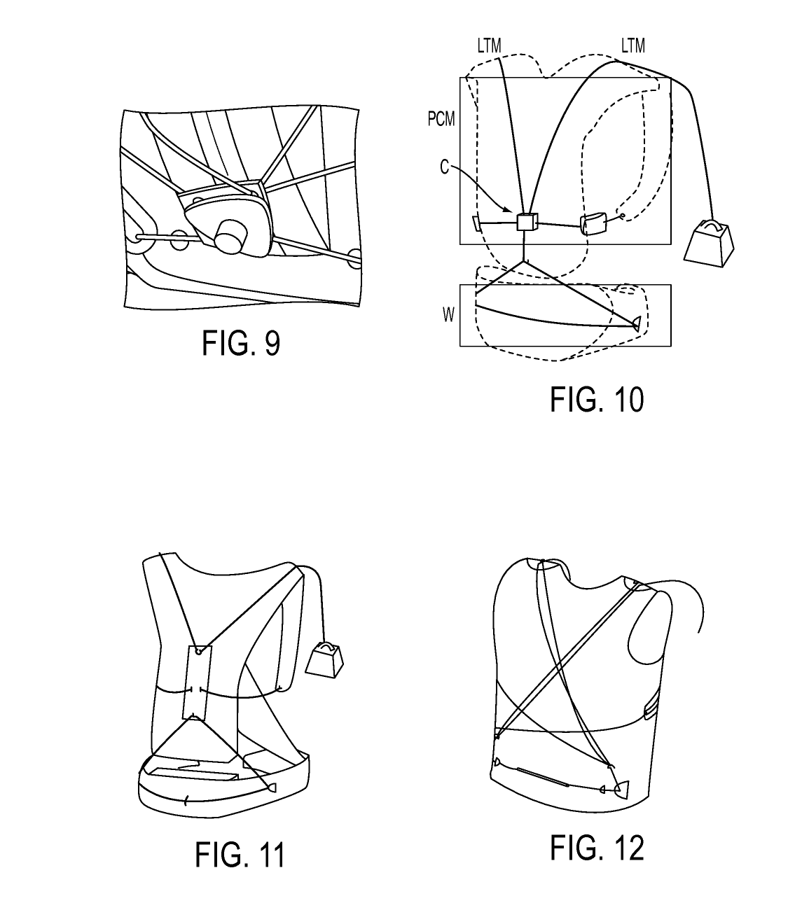

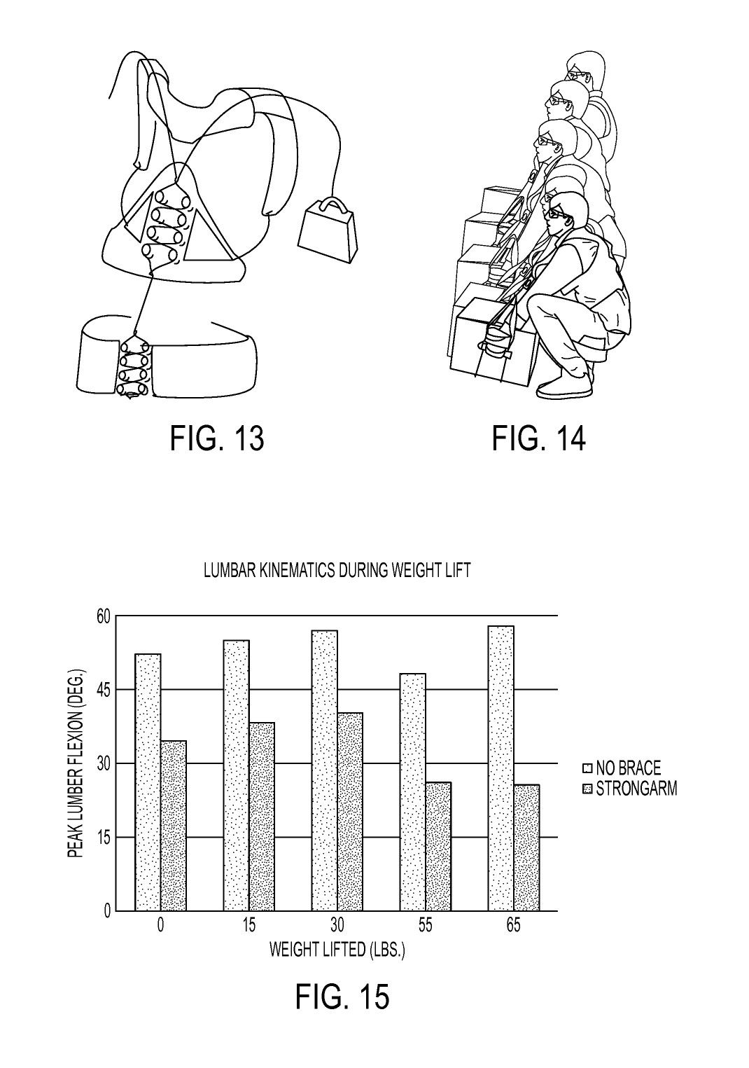

FIG. 7 shows a different embodiment of the invention with the LTM, PCM and C means described above. In this embodiment there is a single coupling means C configured to slide up and down along a channeled slider SL that is placed approximately mid-torso over the spine; as this coupling means C ascends the channel as a result of the downward motion of the load-transfer means LTM at their attachment points to the load (show in the figure as a rectangular weight with a handle attached to the “right” LTM (i.e., the LTM that descends to the wearer's right hand); the corresponding weight on the left LTM is not shown), the postural compliance means PCM compresses the torso—in this embodiment via the drawing in of the shoulder straps—to the appropriate lifting posture. FIG. 8 provides a photograph of a prototype of the embodiment of FIG. 7; FIG. 9 provides an exploded view of the coupling means C in the channeled slider SL of this embodiment. FIGS. 10-13 show additional exemplary embodiments of the present invention, all of which embody the same basic principle of coupling between lifting and the PCM via a single coupling means or multiple coupling means.

As discussed above, as loading on the load-transfer means LTM increases, so too does the postural compliance exerted by the postural compliance means PCM, with the coupling between the two obtained by at least one coupling means C. FIG. 14 provides a super-positioning of images obtained at various stages during the lifting process of a user wearing one embodiment of the present invention; as this figure shows, the back remains in appropriate posture throughout lifting, with a gradated change in posture during lifting to preserve the appropriate posture.

With regard to the change in the postural compliance enforced by the PCM, this change can be linear, or it can be non-linear. Thus for example the PCM may be gradually engaged via increased tensioning of straps as in the embodiment of, e.g., FIG. 5; alternatively the PCM may be designed so that the PCM engages as an full-off or full-on process when sufficient lifting weight in the LTM.

### Measured Ergonomic Effects of the Lift-Assistance Device

As already discussed, in 1994 a “Back Belt Working Group” of the National Institute of Occupational Health and Safety (“NIOSH”) reviewed commercially available lifting belts and concluded that there were insufficient data to indicate “that typical industrial back belts significantly reduce the biomechanical loading of the trunk during manual lifting.” For the present lift-assistance device such data have been obtained; as the exemplary data in FIG. 15 show, this device has a significant effect on reducing peak lumbar flexion during lifting, with the reduction seen for heavier loads reaching over 50%. Thus these data can be used to define one embodiment of the invention, where the sequence of “appropriate lifting postures” that occur with engagement of the PCM are such as to reduce peak lumbar flexion at any particular weight at least to the extent shown in FIG. 15.

### “Lifting Point” Embodiments

As already discussed, LTM generically refers to the means of the present invention for transferring the load weighting from the “lifting point” (hands, wrists, forearms, etc.) over the shoulders and down to the lower torso. Further with regard to specific terminology, at the lifting point on each arm different “lift coupling means” (“LCM”) may be used to couple the LTM ends such as string or strap or wire ends to a hook, glove or other lift coupling means that serves to transfer the weight of the object(s) to be lifted directly to the LTM. Two examples of such LCM are shown in FIGS. 16 and 17; specifically, FIG. 16 shows LCM in the form of gloves that attach to the LTM via a hook, loop, grommet, etc., while FIG. 17 shows attachment to lifting hooks instead of gloves.

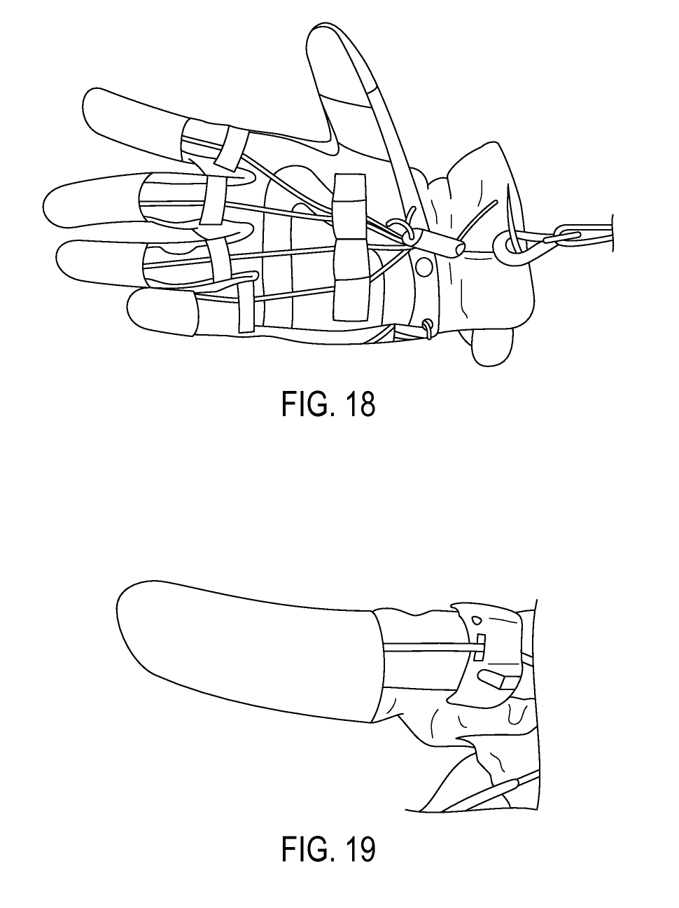

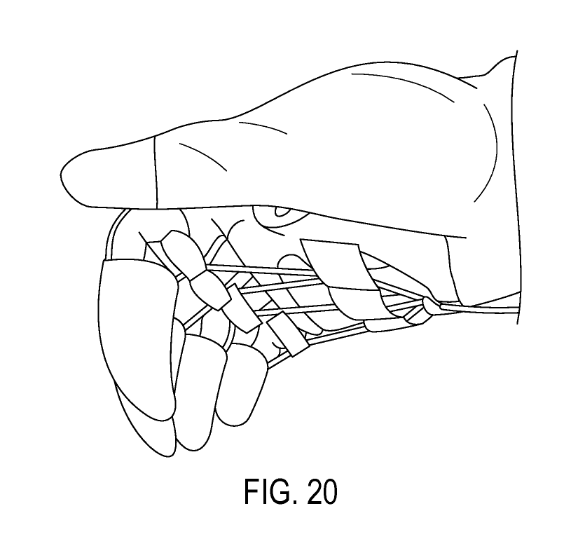

With regard to the various LCM embodiments of the present invention, another non-limiting embodiment of an LCM is the “load-activated grip-assisting glove” embodiment provided in FIGS. 18-20. In this LCM embodiment, a specially-designed glove is intended to increase users grip strength when picking up a load. The device has linear members that run from the fingertips and end at an attachment point at the wrist. The device is worn like a glove and it attaches to the wearer's arm or body, in this case the postural conformance device via the attachment point. When the wearer lifts a load, the forces of that load force the hand into a grip by pulling the string taught therefore curling the fingers. The pieces on the mid finger and the hard finger tips force the fingers to curl in a specific orientation, e.g. in one preferred but non-limiting embodiment a hook-like shape (e.g., the conformation of the device in FIG. 20 versus FIG. 18).

The present invention contemplates various forms of LCM, each of which may be particularly suited to the needs of a worker in a different work environment. Thus for example hook LCM may be particularly appropriate for a worker lifting small boxes, whereas glove LCM may be more appropriate for workers lifting a variety of oddly sized, hard to grip objects. Other non-limiting examples of LCM include, for example, mechanical or electrical grippers, engagement posts, etc.

Finally, Applicants note that FIG. 17 shows that although the LCM of the present invention can be in the form of free-standing straps, wires, strings, etc., that are not constrained to run along at least some part of the upper and lower arm, in some embodiments (e.g., that of FIG. 17) the LCM are constrained to run along at least some length of the arm. Such an embodiment is preferred in a variety of workplace environments where fouling of the straps/wires/strings of the LCM would occur if these were free-standing.

### Sensor-Laden Lift-Assistance Device

In additional embodiments, the present invention is directed to a lift-assistance device or vest that includes feedback sensors to indicate directly to the user, or by telemetry to a telemetry-storage device or remote telemetry network various data on user lifting.

Thus for example, in one embodiment, the lift-assistance device of the invention includes “unsafe weight” mechanical sensors that trip to indicate to the user that a weight outside of safe-lifting parameters is being lifted. Thus in one non-limiting embodiment, each LTM may have installed in it a mechanical device that, upon sufficient weighting, elongates with a pronounced noise, or that, upon elongation, exposes a colored “weight exceeded” color, or some combination of these indicators, to indicate to the user that the weight being lifted is unsafe for that user. Note that this “unsafe weight” may be a fixed weight, or it may be a weight that varies as a function of time-of-day, amount of weight already lifted by the user over the course of the day or in the last time period, some combination of the above, etc. Although the present invention contemplates unsafe weight sensors as typically being mechanical in nature, sensors that similarly signal unsafe weight using electrical means are also explicitly contemplated.

In other embodiments, the on-vest/on-body sensor(s) may transmit load/elongation data from multiple points on-vest/on-body, where such transmission is either wired or, preferably, wireless (e.g., by Bluetooth) to an on-body recording device, an on-body indicator/retransmission device (e.g., a smartphone application), an off-body receiver network, or some combination of the above. Intermittently- or continuously-transmitted data of this sort may be collected for a variety of purposes, including a) feedback to the vest-wearer regarding appropriate load lifting over the course of the day (e.g, as estimated by one or more algorithms regarding user capacity for additional lifting given previous lifts, time of day, state of body, etc.); b) data collection regarding lifting for correlation with injuries (i.e., to collect data for the development of safer-lifting algorithms); c) data collection for employer implementation of optimized worker lifting (e.g., real-time redistribution of workers based on metrics of each worker's approach to maximum lifting per day, per hour, etc., so that efficiency is maximized while likelihood of worker injuries is minimized by ensuring workers are not being overtasked for lifting). The present invention includes not just the hardware required for such implementations, but also the associated software, including software for a) data acquisition and processing; b) data-mining to extract safe lifting algorithm(s); data processing to coordinate workers, with additional software layers to ensure masking of data or other individual privacy layers to ensure protection of employees from inappropriate employer monitoring, etc.

The following claims provide a non-limiting list of some of the embodiments of the present invention. Other embodiments are presented elsewhere herein.

What is claimed is:

1. A lift-assistance device comprising:
i) a load transfer means (“LTM”), for transferring the load weighting from the lifting point over the shoulders and down to one or more points on the torso;
ii) a postural compliance means (“PCM”), for passively/actively enforcing the appropriate back posture or/and sequence of back postures; and,
iii) a coupling means (“CM”), for coupling increased loading on the load-transfer means into increasing engagement of the postural compliance means.

2. The lift-assistance device of claim 1, where the appropriate back posture for each engagement level of the postural compliance means is one that promotes maintenance of the natural curve of the back at that engagement level of the postural compliance means.

3. The lift-assistance device of claim 1, where the appropriate back posture for each engagement level of the postural compliance means is one that reduces peak lumbar flexion at that engagement level of the postural compliance means.

4. The lift-assistance device of claim 3, where the reduction in peak lumbar flexion at a particular weight lifted is at least as shown in FIG. 15 for that weight.

5. The lift-assistance device of claim 1, where the appropriate back posture for each engagement level of the postural compliance means is one that promotes a measurable reduction in wearer injuries.

6. The lift-assistance device of claim 5, where the measurable reduction in wearer injuries is a measurable reduction in wearer back injuries.

7. The lift-assistance device of claim 1, further comprising a lift coupling means (“LCM”) for each arm, where each LCM transfers at least part of the weight of the load to be lifted to the LTM for that arm.

8. The lift-assistance device of claim 7, where the LCM is selected from the group consisting of gloves, hooks, grippers and gripping strips such as Velcro®.

9. The lift-assistance device of claim 1, where the CM is one or more of the CM exemplified in FIGS. 7-9.

10. The lift-assistance device of claim 1, where increasing engagement of the postural compliance means is linearly related to the weight supported by the LTM.

11. The lift-assistance device of claim 1, where increasing engagement of the postural compliance means is non-linearly related to the weight supported by the LTM.

12. The lift-assistance device of claim 11, where increasing engagement of the postural compliance means is a bi-state engagement from disengaged (state 1) to fully engaged (state 2).

13. The lift-assistance device of claim 1, where the device additionally includes one or more sensor for assaying one or more of the load being lifted, loading at one or more points on the user's body or one or more indicators of strain on the user's body from lifting.

14. The lift-assistance device of claim 13, where the one or more sensors includes one or more unsafe-weight sensors.

15. A method for reducing lifting-related injuries comprising lifting while wearing a lift-assistance device comprising:
i) a load transfer means (“LTM”), for transferring the load weighting from the lifting point over the shoulders and down to one or more points on the torso;
ii) a postural compliance means (“PCM”), for passively/actively enforcing the appropriate back posture or/and sequence of back postures; and,
iii) a coupling means (“CM”), for coupling increased loading on the load-transfer means into increasing engagement of the postural compliance means.

16. The lift-assistance device of claim 15, where the appropriate back posture for each engagement level of the postural compliance means is one that reduces peak lumbar flexion at that engagement level of the postural compliance means.

17. The lift-assistance device of claim 16, where the reduction in peak lumbar flexion at a particular weight lifted is at least as shown in FIG. 15 for that weight.

18. The lift-assistance device of claim 15, where the appropriate back posture for each engagement level of the postural compliance means is one that promotes a measurable reduction in wearer injuries.

19. The lift-assistance device of claim 15, further comprising a lift coupling means (“LCM”) for each arm, where each LCM transfers at least part of the weight of the load to be lifted to the LTM for that arm.

20. The lift-assistance device of claim 19, where the LCM is selected from the group consisting of gloves, hooks, grippers and gripping strips such as Velcro®.

21. The lift-assistance device of claim 15, where the CM is one or more of the CM exemplified in FIGS. 7-9.
